(19) United States
(12) Patent Application Publication
(10) Pub. No.: US 2022/0386793 A1
(43) Pub. Date: Dec. 8, 2022

(54) INTELLIGENT NURSING MIRROR, INTELLIGENT NURSING DEVICE, AND IMAGE DISPLAY METHOD AND SYSTEM

(71) Applicant: BOE TECHNOLOGY GROUP CO., LTD., Beijing (CN)

(72) Inventors: Qingguo Yang, Beijing (CN); Honglei Zhang, Beijing (CN); Wenchu Dong, Beijing (CN)

(73) Assignee: BOE TECHNOLOGY GROUP CO., LTD., Beijing (CN)

(21) Appl. No.: 17/775,457

(22) PCT Filed: Jun. 8, 2021
(86) PCT No.: PCT/CN2021/098789

§ 371 Date: May 9, 2022

(30) Foreign Application Priority Data
Jun. 17, 2020 (CN) 202010553875.X

(51) Int. Cl. A47G 1/02; H04N 7/18; G16H 10/60

(52) U.S. Cl. CPC A47G 1/02; H04N 7/188; G16H 10/60

(57) ABSTRACT

An intelligent caring mirror, an intelligent caring device, and an image displaying method and system, which relates to the technical field of image displaying. The intelligent caring mirror (10) includes a imaging unit (101), a processor (102) and a mirror-body displaying assembly (103); the imaging unit (101) is configured for collecting a target image in front of the intelligent caring mirror (10), wherein the target image includes at least a user image and an intelligent-caring-device image; the processor (102) is configured for, according to the user image and the intelligent-caring-device image, acquiring a caring instructing video; and the mirror-body displaying assembly (103) is configured for displaying the caring instructing video.

### CROSS REFERENCE TO RELEVANT APPLICATIONS

The present disclosure claims the priority of the Chinese patent application filed on Jun. 17, 2020 before the Chinese Patent Office with the application number of 202010553875.X and the title of “INTELLIGENT NURSING MIRROR, INTELLIGENT NURSING DEVICE, AND IMAGE DISPLAY METHOD AND SYSTEM”, which is incorporated herein in its entirety by reference.

### TECHNICAL FIELD

The present disclosure relates to the technical field of image displaying technologies, and particularly relates to an intelligent caring mirror, an intelligent caring device, and an image displaying method and system.

### BACKGROUND

The personal caring with long-term such as tooth brushing is beneficial to people's health. and the actual effect of caring to the health of users is closely related to the users' caring mode, and non-standard caring modes are adverse to the users' health.

### SUMMARY

The embodiments of the present disclosure provide an intelligent caring mirror, an intelligent caring device, and an image displaying method and system.

The embodiments of the present disclosure employ the following technical solutions:

In an aspect, there is provided an intelligent caring mirror, wherein the intelligent caring mirror comprises a imaging unit, a processor and a mirror-body displaying assembly;

the imaging unit is configured for collecting a target image in front of the intelligent caring mirror, wherein the target image comprises at least a user image and an intelligent-caring-device image;

the processor is configured for, according to the user image and the intelligent-caring-device image, acquiring a caring instructing video; and

the mirror-body displaying assembly is configured for displaying the caring instructing video.

Optionally, the mirror-body displaying assembly comprises a transparent substrate layer, a transmitting-and-reflecting layer and a displaying layer that are arranged in stack, wherein the transmitting-and-reflecting layer and the displaying layer are located on two sides of the transparent substrate layer;

the transmitting-and-reflecting layer comprises a transmitting-and-reflecting region and a reflecting region, and a light-emitting face of the displaying layer faces the transmitting-and-reflecting region;

the reflecting region is used for reflecting an incident light pointing to the light-emitting face; and

the transmitting-and-reflecting region is used for partially transmitting and partially reflecting of the incident light pointing to the light-emitting face, and transmitting a light exiting from the light-emitting face.

Optionally, a padding layer is provided between the transmitting-and-reflecting layer and the displaying layer.

Optionally, the intelligent caring mirror further comprises:

a first communication element configured for, in response to the intelligent caring device being an intelligent toothbrush, acquiring a caring record of a user from the intelligent toothbrush;

the mirror-body displaying assembly is further configured for displaying the caring record of the user; and

the caring record contains at least one of a current caring duration, a successive-caring-day quantity and a caring score.

Optionally, the first communication element is a first near-field-communication element;

the first near-field-communication element is configured for, in response to a request of communicative connection by a second near-field-communication element of the intelligent toothbrush, performing communicative connection; and

the processor is further configured for, in response to the communicative connection between the first near-field-communication element and the second near-field-communication element of the intelligent toothbrush, controlling the imaging unit to perform image acquisition.

Optionally, the intelligent caring mirror further comprises:

an audio inputting element configured for collecting a voice information of the user; and

the processor is further configured for, if the voice information satisfies a voice-awakening standard, controlling the imaging unit to perform image acquisition.

Optionally, the intelligent caring mirror further comprises:

an audio outputting element configured for outputting voice data of the caring instructing video.

Optionally, the intelligent caring mirror further comprises: an infrared sensor configured for collecting a thermal-radiation information of a current environment; and

the processor is further configured for, in response to the thermal-radiation information of the current environment containing a human-body thermal-radiation information, controlling the imaging unit to perform image acquisition.

In another aspect, there is provided an intelligent caring device, wherein the intelligent caring device comprises:

a main toothbrush body; and

a second communication element configured for sending a caring record of a user to a first communication element of an intelligent caring mirror.

Optionally, the main toothbrush body is provided with at least one identification mark at a surface.

In yet another aspect, there is provided an image displaying method, applied to the intelligent caring mirror stated above, wherein the method comprises:

collecting the target image in front of the intelligent caring mirror, wherein the target image comprises at least a user image and an intelligent-caring-device image;

according to the user image and the intelligent-caring-device image, acquiring the caring instructing video; and

displaying the caring instructing video.

Optionally, the caring instructing video comprises a personal virtual image containing a teeth virtual image, and an intelligent-caring-device virtual image.

Optionally, the step of acquiring the caring instructing video comprises:

acquiring a first relative position of the intelligent-caring-device image and the user image in the target image;

according to the first relative position and a predetermined caring instructing rule, acquiring a second relative position of the personal virtual image and the intelligent-caring-device virtual image; and

according to the second relative position, synthesizing the personal virtual image and the intelligent-caring-device virtual image, to obtain the caring instructing video.

Optionally, the teeth virtual image contains an emphatically displayed mark, and the step of, according to the second relative position, synthesizing the personal virtual image and the intelligent-caring-device virtual image, to obtain the caring instructing video comprises:

according to the second relative position, determining a region of coincidence of a preset continuous duration between the teeth virtual image and the intelligent-caring-device virtual image as a target region;

deleting the emphatically displayed mark of the target region in the teeth virtual image, to obtain the teeth virtual image obtained after the deletion; and

according to the second relative position, synthesizing the personal virtual image containing the teeth virtual image obtained after the deletion and the intelligent-caring-device virtual image, to obtain the caring instructing video.

Optionally, after the step of displaying the caring instructing video, the method further comprises:

collecting a caring image of the user;

acquiring a first position of the intelligent-caring-device image in the caring image of the user, and a second position of the intelligent-caring-device virtual image in the caring instructing video;

comparing the first position and the second position, to obtain a coinciding degree and a coinciding duration of the intelligent-caring-device image and the intelligent-caring-device virtual image; and

according to the coinciding degree and the coinciding duration, obtaining a caring score, and displaying the caring score.

In still another aspect, there is provided an image displaying system, wherein the system comprises the intelligent caring mirror stated above and an intelligent caring device, and the intelligent caring device comprises:

a main toothbrush body; and

a second communication element configured for sending a caring record of a user to a first communication element of the intelligent caring mirror.

Optionally, the main toothbrush body is provided with at least one identification mark at a surface.

In still another aspect, there is provided a calculating and processing device, wherein the calculating and processing device comprises:

a memory storing a computer-readable code; and

one or more processors, wherein when the computer-readable code is executed by the one or more processors, the calculating and processing device implements the image displaying method stated above.

In still another aspect, there is provided a computer program product, wherein the computer program product comprises a computer-readable code, and when the computer-readable code is executed in a calculating and processing device, the computer-readable code causes the calculating and processing device to implement the image displaying method stated above.

In still another aspect, there is provided a non-transitory computer-readable storage medium, wherein the non-transitory computer-readable storage medium stores a computer instruction, and the computer instruction, when executed by a processor, implements the image displaying method stated above.

The above description is merely a summary of the technical solutions of the present disclosure. In order to more clearly know the elements of the present disclosure to enable the implementation according to the contents of the description, and in order to make the above and other purposes, features and advantages of the present disclosure more apparent and understandable, the particular embodiments of the present disclosure are provided below.

### BRIEF DESCRIPTION OF THE DRAWINGS

In order to more clearly illustrate the technical solutions of the embodiments of the present disclosure or the related art, the figures that are required to describe the embodiments or the related art will be briefly introduced below. Apparently, the figures that are described below are merely embodiments of the present disclosure, and a person skilled in the art can obtain other figures according to these figures without paying creative work.

FIG. 1 is a schematic structural diagram of the intelligent caring mirror according to an embodiment of the present disclosure;

FIG. 2 is a schematic structural diagram of the mirror-body displaying assembly according to an embodiment of the present disclosure;

FIG. 3 is a schematic structural diagram of the mirror-body displaying assembly according to another embodiment of the present disclosure;

FIG. 4 is a schematic structural diagram of the intelligent caring mirror according to another embodiment of the present disclosure;

FIG. 5 is a schematic structural diagram of the intelligent caring device according to an embodiment of the present disclosure;

FIG. 6 is a flow chart of the steps of the image displaying method according to an embodiment of the present disclosure;

FIG. 7 is a flow chart of the steps of the video acquiring method according to an embodiment of the present disclosure;

FIG. 8 is a flow chart of the steps of the video acquiring method according to another embodiment of the present disclosure;

FIG. 9 is a flow chart of the steps of a caring assessing method in the related art;

FIG. 10 schematically shows a block diagram of a calculating and processing device for implementing the method according to the present disclosure; and

FIG. 11 schematically shows a storage unit for maintaining or carrying a program code for implementing the method according to the present disclosure.

### DETAILED DESCRIPTION

The technical solutions of the embodiments of the present disclosure will be clearly and completely described below with reference to the drawings of the embodiments of the present disclosure. Apparently, the described embodiments are merely certain embodiments of the present disclosure, rather than all of the embodiments. All of the other embodiments that a person skilled in the art obtains on the basis of the embodiments of the present disclosure without paying creative work fall within the protection scope of the present disclosure.

In the embodiments of the present disclosure, terms such as “first” and “second” are used to distinguish identical items or similar items that have substantially the same functions and effects, merely in order to clearly describe the technical solutions of the embodiments of the present disclosure, and should not be construed as indicating or implying the degrees of importance or implicitly indicating the quantity of the specified technical features.

In the embodiments of the present disclosure, the terms that indicate orientation or position relations, such as “inner” and “outer”, are based on the orientation or position relations shown in the drawings, and are merely for conveniently describing the present disclosure and simplifying the description, rather than indicating or implying that the device or element must have the specific orientation and be constructed and operated according to the specific orientation. Therefore, they should not be construed as a limitation on the present disclosure.

In the embodiments of the present disclosure, the meaning of “plurality of times” is “two or more times”, unless explicitly and particularly defined otherwise.

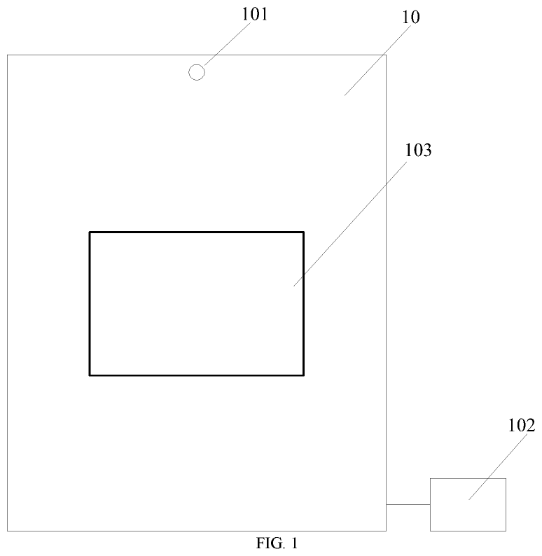

Referring to FIG. 1, FIG. 1 shows an intelligent caring mirror 10 according to an embodiment of the present disclosure. The intelligent caring mirror 10 comprises an imaging unit 101, a processor 102 and a mirror-body displaying assembly.

The imaging unit 101 is configured for collecting a target image in front of the intelligent caring mirror 10, wherein the target image comprises at least a user image and an intelligent-caring-device image.

In an embodiment of the present disclosure, the imaging unit 101 may be a functional component with the function of image acquisition. It may be integrated with the mirror-body displaying assembly 103, and may also be provided independently of the mirror-body displaying assembly 103, as long as it can perform image acquisition to the front of the intelligent caring mirror, which is not particularly limited herein.

The target image refers to an image that contains a user image and an intelligent-caring-device image, and is particularly obtained by the user by the imaging unit 101 to perform image acquisition while the user is located in front of the intelligent caring mirror 10. The user image refers to the image of the displayed region where the user is located in the target image. The intelligent-caring-device image refers to the image of the displayed region where the intelligent caring device is located in the target image.

In practical applications, the user may stand in front of the intelligent caring mirror 10 during the caring, thereby triggering the intelligent caring mirror 10 to perform image acquisition to obtain the target image in the process of the user performing caring by the intelligent caring device. The intelligent caring device may be a toothbrush, a dental floss, a facial mask, a skin softer and so on for caring, which may be particularly determined according to practical demands, and is not limited herein.

The processor 102 is configured for, according to the user image and the intelligent-caring-device image, acquiring a caring instructing video.

In an embodiment of the present disclosure, the type of the processor 102 (Central Processing Unit, CPU) is not limited, and it may be a single chip microcomputer, an Advanced Reduced Instruction Set Computing Machine (Advanced RISC Machines, ARM), a Field Programmable Gate Array (FPGA) and so on, which may be particularly determined according to practical design requirements.

The caring instructing video refers to a video containing image contents for instructing the user to use the intelligent caring device. The image contents of the caring instructing video may particularly be determined according to intelligent-caring-device information such as the type and the position of the intelligent caring device in the intelligent-caring-device image, and user information such as the body actions, the user gender and the user age of the user in the user image. It can be understood that, regarding different intelligent caring devices, the image contents of the caring instructing videos required to instruct the user are different, and regarding different types of the user information such as the body actions and the user gender of the user, the image contents of the required caring instructing videos are different. The particular mode of acquiring the caring instructing video according to the user image and the intelligent-caring-device image may be determined according to practical demands, and is not limited herein.

The mirror-body displaying assembly 103 is configured for displaying the caring instructing video.

In an embodiment of the present disclosure, the mirror-body displaying assembly 103 does not only comprise a mirror surface with the function of light-ray reflection, but also comprises a displaying layer with the function of image displaying. The appearance of the mirror-body displaying assembly 103 may be a mirror such as a conventional square mirror, circle mirror and ellipse mirror, but a displaying assembly is integrated in the mirror body, whereby the caring instructing video may be displayed by the mirror surface. The image displaying region of the mirror-body displaying assembly 103 may be the whole mirror-surface region of the mirror-body displaying assembly 103, and may also be part of the mirror-surface region. When the mirror-body displaying assembly 103 is not displaying the caring instructing video, it may be used by the user as a mirror. When the image displaying region of the mirror-body displaying assembly 103 is part of the area of the mirror surface, the region of the mirror surface that is not the image displaying region may be used as a mirror while displaying the caring instructing video.

The intelligent caring mirror according to the present disclosure can, while the user is performing personal caring in front of the intelligent caring mirror, by performing image acquisition to the process of the user performing the caring, and according to the user image and the intelligent-caring-device image, display the caring instructing video, instructs the users' caring using the intelligent caring device, whereby the user can conveniently obtain the instruction for the caring during the caring.

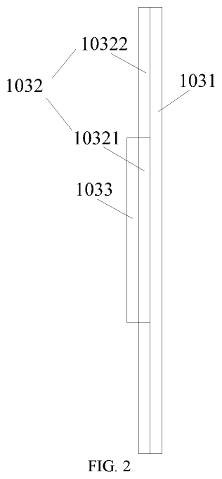

Optionally, referring to FIG. 2, the mirror-body displaying assembly 103 comprises a transparent substrate layer 1031, a transmitting-and-reflecting layer 1032 and a displaying layer 1033 that are arranged in stack. The transmitting-and-reflecting layer 1032 and the displaying layer 1033 are located on the two sides of the transparent substrate layer 1031. The transmitting-and-reflecting layer 1032 comprises a transmitting-and-reflecting region 10321 and a reflecting region 10322, and the light-emitting face of the displaying layer 1033 faces the transmitting-and-reflecting region 10321. The reflecting region 10322 is used for reflecting an incident light pointing to the light-emitting face. The transmitting-and-reflecting region 10321 is used for partially transmitting and partially reflecting of the incident light pointing to the light-emitting face, and transmitting a light exiting from the light-emitting face.

In an embodiment of the present disclosure, the transparent substrate layer 1031 may use a light transmitting material such as a transparent glass. The reflecting region 10322 of the transmitting-and-reflecting layer 1032 may use a light tight material such as a silver powder, which can reflect all of the light rays emitted in front of the intelligent caring mirror 10. The transmitting-and-reflecting region 10321 of the transmitting-and-reflecting layer 1032 may be a semi-transparent film having a certain capacity of light transmission, whereby some of the incident light rays can be reflected and the other incident light rays can transmit the transmitting-and-reflecting region 10321 of the transmitting-and-reflecting layer 1032, wherein the particular proportion between the reflected light and the transmitted light may be determined according to practical demands, and is not limited herein. The displaying layer 1033 is a display screen adhesively bonded to the transmitting-and-reflecting region 10321 of the transmitting-and-reflecting layer 1032. The type of the displaying layer is not limited, it may be a semi-conductor-process display screen such as a Liquid-Crystal Display (LCD) screen, an Organic Light Emitting Diode (OLED) screen, and a Micro Light Emitting Diode (Micro LED) screen and may also be a projection screen, an LED screen and so on, and may be a flat screen or a curved screen, as long as it can perform image displaying. The displaying layer 1033 and the transparent substrate layer 1031 are adhesively bonded to the two opposite faces of the transmitting-and-reflecting layer 1032.

Particularly, the transparent substrate layer 1031 is adhesively bonded to the reflecting region 10322 of the transmitting-and-reflecting layer 1032, and, in the mirror-surface region of the transparent substrate layer 1031 that is adhesively bonded to the reflecting region 10322 of the transmitting-and-reflecting layer 1032, because the reflecting region 10322 of the transmitting-and-reflecting layer 1032 can totally reflect the light rays, the first minor-surface region where the reflecting layer is located may be used as a minor. Moreover, because the transmitting-and-reflecting layer 1032 has the function of semi-transmission, while the displaying layer 1033 is not performing image displaying, the second minor-surface region of the transmitting-and-reflecting layer 1032 and the transparent substrate layer 1031 may be used as a mirror, and while the displaying layer 1033 is performing image displaying, the transmitting layer may present the image displayed by the displaying layer 1033 via the transparent substrate layer 1031.

In the embodiments of the present disclosure, by providing the transmitting-and-reflecting layer at the intelligent caring minor with the reflecting region and the transmitting-and-reflecting region, and providing the displaying layer behind the transmitting-and-reflecting layer, as compared with minors in the related art with merely a reflecting layer and a glass layer, the intelligent caring minor cannot only be used as a minor, but can also, while the user is performing caring in front of the intelligent caring minor, enable the user to conveniently watch the caring instructing video by the intelligent caring mirror.

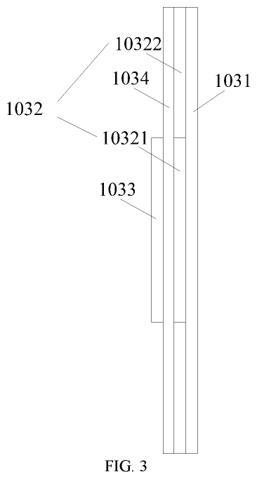

Optionally, referring to FIG. 3, the transparent substrate layer 1031 is a glass layer. A padding layer 1034 is provided between the transmitting-and-reflecting layer 1032 and the displaying layer 1033.

In an embodiment of the present disclosure, the padding layer 1034 is a light transmitting material with the characteristics such as a thermal conductivity and an electric conductivity, for example, single crystal, silicon carbide and so on, which is not limited herein. The embodiment of the present disclosure, by providing the padding layer between the transmitting layer and the displaying layer, the effect of frame displaying of the displaying layer is effectively improved.

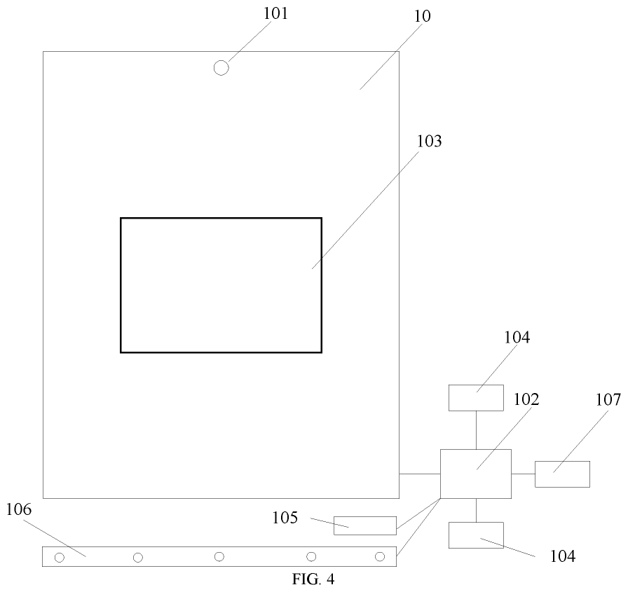

Optionally, referring to FIG. 4, the intelligent caring mirror 10 further comprises:

a first communication element 104 configured for, when the intelligent caring device is an intelligent toothbrush, acquiring a caring record of a user from the intelligent toothbrush.

In an embodiment of the present disclosure, the first communication element 104 may be a functional component with the function of wireless data transmission. The function of wireless data transmission may be implemented by, for example, Bluetooth, a wireless network, a mobile network, near-field communication (NFC) and so on, which is not limited herein. The first communication element 104 may be communicatively connected to the second communication element of the intelligent toothbrush, thereby acquiring the caring record of the user.

The caring record contains at least one of a current caring duration, a successive-caring-day quantity and a caring score.

In an embodiment of the present disclosure, the current caring duration refers to the continuous duration of the users' caring this time. The successive-caring-day quantity refers to the continuous days of caring performed by the user every day. The caring score refers to a evaluation score of the uses' caring, and may be determined according to the parameters of the users' caring such as the body actions, the duration and the frequency, which is not limited herein.

The mirror-body displaying assembly 103 is further configured for displaying the caring record of the user.

In an embodiment of the present disclosure, the mirror-body displaying assembly 103 may also display the caring record acquired from the intelligent toothbrush, whereby the user can know the profile of the caring in real time by the intelligent caring mirror.

Optionally, the first communication element 104 is a first near-field-communication element; and

the first near-field-communication element is configured for being communicatively connected to a second near-field-communication element of the intelligent toothbrush.

In an embodiment of the present disclosure, the first near-field-communication element and the second near-field-communication element refer to NFC, whereby, when the distance between the intelligent toothbrush and the intelligent caring mirror is less than, for example, 1 meter or 50 centimeters, the intelligent toothbrush and the intelligent caring mirror can be communicatively connected by the first near-field-communication element and the second near-field-communication element.

The processor 102 is further configured for, when the first near-field-communication element is communicatively connected to the second near-field-communication element of the intelligent toothbrush, controlling the imaging unit to perform image acquisition.

In an embodiment of the present disclosure, when the user approaches the intelligent caring mirror 10 while holding the intelligent toothbrush in hand, the first near-field-communication element of the intelligent caring mirror 10 automatically establishes communicative connection with the second near-field-communication element of the intelligent toothbrush, at which point the processor 102 automatically electrifies the intelligent caring mirror 10, and controls the imaging unit 101 to perform image acquisition to the front of the intelligent caring mirror.

In the embodiment of the present disclosure, by providing NFC in the intelligent caring mirror, when the user approaches the intelligent caring mirror while holding the intelligent toothbrush in hand, the intelligent caring mirror can be automatically awakened to perform image acquisition, whereby the user can conveniently acquire the caring instructing video.

Optionally, referring to FIG. 4, the intelligent caring mirror 10 further comprises:

an audio inputting element 105 configured for collecting a voice information of the user.

In an embodiment of the present disclosure, the audio inputting element 105 is a functional component with the functions of audio collection and recording, for example, a microphone and so on, and can collect the voice information of the user.

The processor 102 is further configured for, if the voice information satisfies a voice-awakening standard, controlling the imaging unit to perform image acquisition.

In an embodiment of the present disclosure, the voice-awakening standard may be a judging mechanism that contains standards such as the target voice content and the target voice duration, and may determine, according to the contents contained in the voice information of the user, whether it is required to awaken the intelligent caring mirror 10. The processor 102 may identify the voice information of the user by a pre-provided voice recognition model, and, by using the parameters such as the contents and the duration of the voice information, according to the voice-awakening standard, it is determined whether to awaken the intelligent caring mirror. For example, when the collected voice information of the user is contents such as “Magic mirror” and “I want to brush my teeth”, the intelligent caring mirror is awakened.

In the embodiment of the present disclosure, by awakening the intelligent caring mirror by using the voice information of the user, the user can conveniently acquire the caring instructing video by using the intelligent caring mirror.

Optionally, referring to FIG. 4, the intelligent caring mirror 10 further comprises:

an audio outputting element 106 configured for outputting voice data of the caring instructing video.

In an embodiment of the present disclosure, the audio outputting element 106 may be a functional component with the functions of audio playing and audio transmission. The audio outputting element 106 may directly play the voice data of the caring instructing video, and the voice data of the caring instructing video may also be sent to and played by another audio playing device, such as an earphone or a loudspeaker of the user, which may be particularly determined according to practical demands, and is not limited herein.

Optionally, referring to FIG. 4, the intelligent caring mirror 10 further comprises: an infrared sensor 107 configured for collecting a thermal-radiation information of a current environment.

In an embodiment of the present disclosure, the infrared sensor 107 may be a sensor with the function of infrared-ray sensing. In order to improve the effect of the infrared sensor 107, an infrared probe may be provided in the infrared sensor 107 to acquire the thermal-radiation information of the current environment in front of the intelligent caring mirror, which may be particularly determined according to practical demands, and is not limited herein.

The processor 102 is further configured for, when the thermal-radiation information of the current environment contains a human-body thermal-radiation information, controlling the imaging unit to perform image acquisition.

In an embodiment of the present disclosure, by human-body thermal radiation analysis on the thermal-radiation information of the current environment collected by the infrared sensor 107, it is determined whether the current environment contains a human-body heat source, or, in other words, whether the thermal-radiation information of the current environment contains the human-body thermal-radiation information, to determine whether the user is approaching the intelligent caring mirror 10. If it is determined that the current environment contains a human-body heat source, the processor 102 awakens the intelligent caring mirror.

In the embodiment of the present disclosure, by automatically awakening the intelligent caring mirror when the infrared sensor detects that the user approaches the intelligent caring mirror, to provide the caring instructing video to the user, the user can more conveniently acquire the caring instructing video by using the intelligent caring mirror.

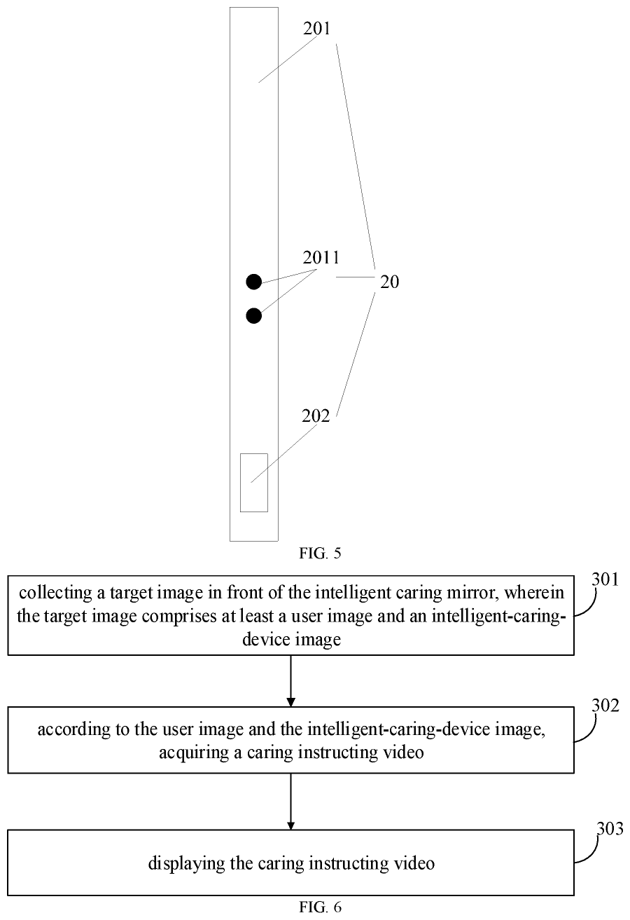

Referring to FIG. 5, FIG. 5 shows an intelligent caring device 20 according to an embodiment of the present disclosure. The intelligent caring device 20 comprises a main toothbrush body 201 and a second communication element 202.

In an embodiment of the present disclosure, the main toothbrush body 201 comprises the structural components of conventional toothbrushes such as the brush hair and the brush rod. The material of the main toothbrush body 201 may be wood, rubber and so on, which is not limited herein.

The second communication element 202 is configured for sending a caring record of a user to a first communication element of the intelligent caring mirror.

In an embodiment of the present disclosure, the second communication element 202 may be a functional component with the function of wireless data transmission. The function of wireless data transmission may be implemented by using, for example, Bluetooth, a wireless network, a mobile network, near-field communication (NFC) and so on, which is not limited herein.

The intelligent caring device provided the embodiment of the present disclosure, sends the caring record of the user to the intelligent caring mirror by using the second communication element, so that the user can know the profile of his caring in real time by using the intelligent caring mirror.

Optionally, referring to FIG. 5, the main toothbrush body 201 is provided with at least one identification mark 2011 at the surface.

In an embodiment of the present disclosure, the identification mark may be a mark of specific color and shape, for example, a red circular dot, a black square and so on. The intelligent caring mirror of the user identifies the intelligent caring device, thereby obtaining the data processing quantity of the intelligent caring mirror.

Referring to FIG. 6, an image displaying method according to an embodiment of the present disclosure is shown, applied to the intelligent caring mirror according to the above embodiments. The method comprises:

Step 301: collecting a target image in front of the intelligent caring mirror, wherein the target image comprises at least a user image and an intelligent-caring-device image.

Step 302: according to the user image and the intelligent-caring-device image, acquiring a caring instructing video.

Step 303: displaying the caring instructing video.

The step 301 to the step 302 and the embodiments may refer to the detailed description on the intelligent caring mirror in the embodiments, which, in order to avoid replication, is not discussed here further.

The image displaying method according to the present disclosure can, while the user is performing caring in front of the intelligent caring mirror, by performing image acquisition to the process of the user performing the caring, and according to the user image and the intelligent-caring-device image, display the caring instructing video, instructs the users' caring by the intelligent caring device, whereby the user can conveniently obtain the instruction for the caring during the caring.

Optionally, the caring instructing video comprises a personal virtual image containing a teeth virtual image, and an intelligent-caring-device virtual image.

In an embodiment of the present disclosure, the teeth virtual image is an image simulating the teeth of the user in the personal virtual image, and may be displayed emphatically by highlighting, enlargement and so on.

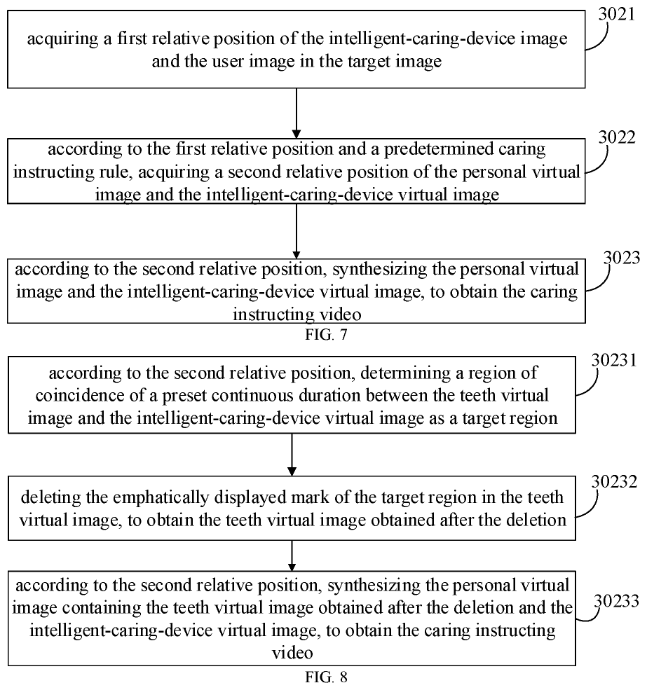

Referring to FIG. 7, the step 302 comprises:

Sub-step 3021: acquiring a first relative position of the intelligent-caring-device image and the user image in the target image.

In an embodiment of the present disclosure, the first relative position of the intelligent-caring-device image and the user image refers to the relative position of the intelligent-caring-device image and the user image in the target image, and may particularly comprise the relative distance and the image coordinates.

Sub-step 3022: according to the first relative position and a predetermined caring instructing rule, acquiring a second relative position of the personal virtual image and the intelligent-caring-device virtual image.

In an embodiment of the present disclosure, the predetermined caring instructing rule refers to a rule that is made in advance according to normal caring modes, for example, when the intelligent caring device is a toothbrush, rules with respect to the position, the sequence and the duration of the tooth brushing on the teeth. The personal virtual image and the intelligent-caring-device virtual image refer to 3D images, animation images and so on that simulate respectively the user and the intelligent caring device, which is not limited herein. The second relative position refers to the relative position of the intelligent-caring-device virtual image and the user virtual image in the required caring instructing video.

By using the predetermined caring instructing rule, and based on the first relative position of the intelligent-caring-device image and the user image, the content that requires to be instructed the user how to perform caring in the next step can be determined, i.e., how the user is required to move the intelligent caring device.

Sub-step 3023: according to the second relative position, synthesizing the personal virtual image and the intelligent-caring-device virtual image, to obtain the caring instructing video.

In an embodiment of the present disclosure, the personal virtual image and the intelligent-caring-device virtual image may synthesized according to the second relative position, to obtain the caring instructing video.

In the embodiment of the present disclosure, by displaying a suitable caring instructing video to the user, according to the relative position of the intelligent caring device and the user in the process of caring, the displayed caring instructing video can satisfy the practical demands of the user better, which improves the effectiveness of the caring instruction.

Optionally, the teeth virtual image contains an emphatically displayed mark, and, referring to FIG. 8, the sub-step 3023 comprises:

Sub-step 30231: according to the second relative position, determining a region of coincidence of a preset continuous duration between the teeth virtual image and the intelligent-caring-device virtual image as a target region.

In an embodiment of the present disclosure, the emphatically displayed mark refers to a mark image that simulates patterns such as a dental plaque, a tartar and a blot, and is used to prompt the user which parts still require to be cared.

In practical applications, the caring instructing video changes in real time according to the positions of the user image and the intelligent-caring-device image. The caring instructing video instructs the user to care sequentially the areas of the teeth, and after the contents of the caring instructing video have already instructed the user to care a certain area of teeth, it is required to inform the user that the area has already been cared. Particularly, a region of coincidence of a preset continuous duration between the teeth virtual image and the intelligent-caring-device virtual image in the caring instructing video may be determined as a target region that has already been cared. The preset duration may be, for example, 2 s, 3 s and so on, which may be particularly determined according to practical demands, and is not limited herein.

Sub-step 30232: deleting the emphatically displayed mark of the target region in the teeth virtual image, to obtain the teeth virtual image obtained after the deletion.

In an embodiment of the present disclosure, the emphatically displayed mark in the target region determined in the teeth virtual image is erased, and accordingly a teeth virtual image that can prompt the user the area that has already been cared can be obtained.

Sub-step 30233: according to the second relative position, synthesizing the personal virtual image containing the teeth virtual image obtained after the deletion and the intelligent-caring-device virtual image, to obtain the caring instructing video.

In an embodiment of the present disclosure, by synthesizing the user virtual image and the intelligent-caring-device virtual image in which the emphatically displayed mark has been deleted, the caring instructing video that can prompt the user the area of the teeth that has already been cared can be obtained, which improves the enjoyment of the caring instruction.

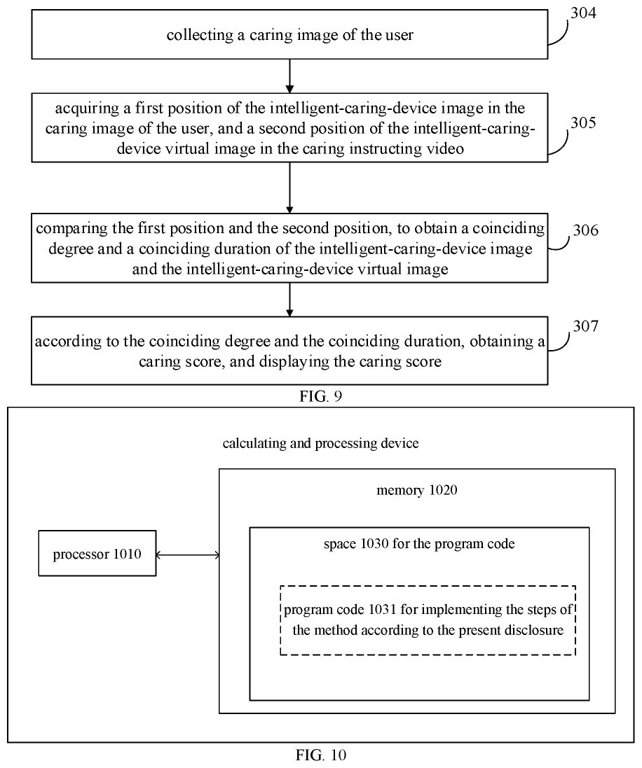

Optionally, referring to FIG. 9, after the step 303, the method further comprises:

Step 304: collecting a caring image of the user.

In an embodiment of the present disclosure, the caring image of the user refers to an image of the process of the user performing caring, and is particularly collected after the caring instructing video has been displayed.

Step 305: acquiring a first position of the intelligent-caring-device image in the caring image of the user, and a second position of the intelligent-caring-device virtual image in the caring instructing video.

In an embodiment of the present disclosure, the first position represents the position of the intelligent caring device in the process of the user actually performing the caring, and the second position represents the position of the intelligent caring device in the displayed caring instructing video.

Step 306: comparing the first position and the second position, to obtain a coinciding degree and a coinciding duration of the intelligent-caring-device image and the intelligent-caring-device virtual image.

In an embodiment of the present disclosure, when the first position and the second position have been dimension-normalized, the coinciding degree between the first position and the second position, and the total duration during which the coinciding degree is greater than a coinciding-degree threshold, i.e., the coinciding duration, are determined.

Step 307: according to the coinciding degree and the coinciding duration, obtaining a caring score, and displaying the caring score.

In an embodiment of the present disclosure, the behavior of the user performing the caring by referring to the caring instructing video may be scored according to the coinciding degree and the coinciding duration. The scoring mechanism may be a higher coinciding degree results in a higher score, a longer coinciding duration results in a higher score, and vice versa. Certainly, another scoring mechanism may be provided, which may be particularly determined according to practical demands, and is not limited herein.

In the embodiment of the present disclosure, by comparing the actual operation to the intelligent caring device by the user in the process of the caring and the instruction operation of the caring instructing video, the users' caring is scored, whereby the user can conveniently know the profile of his caring, and the enjoyment of the caring instruction is improved.

It should be noted that the relevant structures and the principle of the locating of the intelligent caring mirror and the intelligent caring device described above may refer to the above embodiments, and are not discussed here further.

An embodiment of the present disclosure provides an image displaying system, wherein the image displaying system comprises the intelligent caring mirror and the intelligent caring device according to the above embodiments.

In the interaction system, by configuring the intelligent caring mirror and the intelligent caring device to match each other, not only the user can be provided with the caring instructing video conveniently in the process of the user performing caring, but also the user can be displayed with his own caring record by the data interaction between the intelligent caring device and the intelligent caring mirror, whereby the user can know the profile of the caring in real time.

The above-described device embodiments are merely illustrative, wherein the units that are described as separate components may or may not be physically separate, and the components that are displayed as units may or may not be physical units; in other words, they may be located at the same one location, and may also be distributed to a plurality of network units. Some or all of the modules may be selected according to the actual demands to realize the purposes of the solutions of the embodiments. A person skilled in the art can understand and implement the technical solutions without paying creative work.

Each component embodiment of the present disclosure may be implemented by hardware, or by software modules that are operated on one or more processors, or by a combination thereof. A person skilled in the art should understand that some or all of the functions of some or all of the components of the calculating and processing device according to the embodiments of the present disclosure may be implemented by using a microprocessor or a digital signal processor (DSP) in practice. The present disclosure may also be implemented as apparatus or device programs (for example, computer programs and computer program products) for implementing part of or the whole of the method described herein. Such programs for implementing the present disclosure may be stored in a computer-readable medium, or may be in the form of one or more signals. Such signals may be downloaded from an Internet website, or provided on a carrier signal, or provided in any other forms.

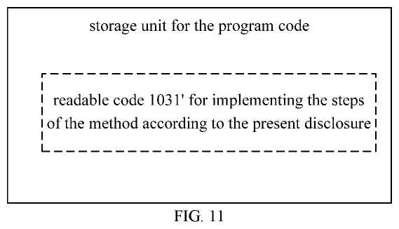

For example, FIG. 10 shows a calculating and processing device that can implement the method according to the present disclosure. The calculating and processing device traditionally comprises a processor 1010 and a computer program product or computer-readable medium in the form of a memory 1020. The memory 1020 may be electronic memories such as flash memory, EEPROM (Electrically Erasable Programmable Read Only Memory), EPROM, hard disk or ROM. The memory 1020 has the storage space 1030 of the program code 1031 for implementing any steps of the above method. For example, the storage space 1030 for program code may contain program codes 1031 for individually implementing each of the steps of the above method. Those program codes may be read from one or more computer program products or be written into the one or more computer program products. Those computer program products include program code carriers such as a hard disk, a compact disk (CD), a memory card or a floppy disk. Such computer program products are usually portable or fixed storage units as shown in FIG. 11. The storage unit may have storage segments or storage spaces with similar arrangement to the memory 1020 of the calculating and processing device in FIG. 10. The program codes may, for example, be compressed in a suitable form. Generally, the storage unit contains a computer-readable code 1031′, which can be read by a processor like 1010. When those codes are executed by the calculating and processing device, the codes cause the calculating and processing device to implement each of the steps of the method described above.

The “one embodiment”, “an embodiment” or “one or more embodiments” as used herein means that particular features, structures or characteristics described with reference to an embodiment are included in at least one embodiment of the present disclosure. Moreover, it should be noted that here an example using the wording “in an embodiment” does not necessarily refer to the same one embodiment.

The description provided herein describes many concrete details. However, it can be understood that the embodiments of the present disclosure may be implemented without those concrete details. In some of the embodiments, well-known processes, structures and techniques are not described in detail, so as not to affect the understanding of the description.

In the claims, any reference signs between parentheses should not be construed as limiting the claims. The word “comprise” does not exclude elements or steps that are not listed in the claims. The word “a” or “an” preceding an element does not exclude the existing of a plurality of such elements. The present disclosure may be implemented by means of hardware comprising several different elements and by means of a properly programmed computer. In unit claims that list several devices, some of those devices may be embodied by the same item of hardware. The words first, second, third and so on do not denote any order. Those words may be interpreted as names.

The above are merely particular embodiments of the present disclosure, and the protection scope of the present disclosure is not limited thereto. All of the variations or substitutions that a person skilled in the art can easily envisage within the technical scope disclosed by the present disclosure should fall within the protection scope of the present disclosure. Therefore, the protection scope of the present disclosure should be subject to the protection scope of the claims.

1. An intelligent caring mirror, wherein the intelligent caring mirror comprises an imaging unit, a processor and a mirror-body displaying assembly;
the imaging unit is configured for collecting a target image in front of the intelligent caring mirror, wherein the target image comprises at least a user image and an intelligent-caring-device image;
the processor is configured for, according to the user image and the intelligent-caring-device image, acquiring a caring instructing video; and
the mirror-body displaying assembly is configured for displaying the caring instructing video.

2. The intelligent caring mirror according to claim 1, wherein the mirror-body displaying assembly comprises a transparent substrate layer, a transmitting-and-reflecting layer and a displaying layer that are arranged in stack, wherein the transmitting-and-reflecting layer and the displaying layer are located on two sides of the transparent substrate layer;
the transmitting-and-reflecting layer comprises a transmitting-and-reflecting region and a reflecting region, and a light-emitting face of the displaying layer faces the transmitting-and-reflecting region;
the reflecting region is used for reflecting an incident light pointing to the light-emitting face; and
the transmitting-and-reflecting region is used for partially transmitting and partially reflecting of the incident light pointing to the light-emitting face, and transmitting a light emitted from the light-emitting face.

3. The intelligent caring mirror according to claim 2, wherein a padding layer is provided between the transmitting-and-reflecting layer and the displaying layer.

4. The intelligent caring mirror according to claim 1, wherein the intelligent caring mirror further comprises:
a first communication element configured for, in response to the intelligent caring device being an intelligent toothbrush, acquiring a caring record of a user from the intelligent toothbrush;
the mirror-body displaying assembly is further configured for displaying the caring record of the user; and
the caring record contains at least one of a current caring duration, a successive-caring-day quantity and a caring score.

5. The intelligent caring mirror according to claim 4, wherein the first communication element is a first near-field-communication element;
the first near-field-communication element is configured for, in response to a request of communicative connection by a second near-field-communication element of the intelligent toothbrush, performing communicative connection; and
the processor is further configured for, in response to the communicative connection between the first near-field-communication element and the second near-field-communication element of the intelligent toothbrush, controlling the imaging unit to perform image acquisition.

6. The intelligent caring mirror according to claim 1, wherein the intelligent caring mirror further comprises:
an audio inputting element configured for collecting a voice information of the user; and
the processor is further configured for, if the voice information satisfies a voice-awakening standard, controlling the imaging unit to perform image acquisition.

7. The intelligent caring mirror according to claim 1, wherein the intelligent caring mirror further comprises:
an audio outputting element configured for outputting voice data of the caring instructing video.

8. The intelligent caring mirror according to claim 1, wherein the intelligent caring mirror further comprises: an infrared sensor configured for collecting a thermal-radiation information of a current environment; and
the processor is further configured for, in response to the thermal-radiation information of the current environment containing a human-body thermal-radiation information, controlling the imaging unit to perform image acquisition.

9. An intelligent caring device, wherein the intelligent caring device comprises:
a main toothbrush body; and
a second communication element configured for sending a caring record of a user to a first communication element of an intelligent caring mirror.

10. The intelligent caring device according to claim 9, wherein the main toothbrush body is provided with at least one identification mark at a surface.

11. An image displaying method, applied to the intelligent caring mirror according to claim 1, wherein the method comprises:
collecting the target image in front of the intelligent caring mirror, wherein the target image comprises at least a user image and an intelligent-caring-device image;
according to the user image and the intelligent-caring-device image, acquiring the caring instructing video; and
displaying the caring instructing video.

12. The method according to claim 11, wherein the caring instructing video comprises a personal virtual image containing a teeth virtual image, and an intelligent-caring-device virtual image.

13. The method according to claim 12, wherein the step of acquiring the caring instructing video comprises:
acquiring a first relative position of the intelligent-caring-device image and the user image in the target image;
according to the first relative position and a predetermined caring instructing rule, acquiring a second relative position of the personal virtual image and the intelligent-caring-device virtual image; and
according to the second relative position, synthesizing the personal virtual image and the intelligent-caring-device virtual image, to obtain the caring instructing video.

14. The method according to claim 13, wherein the teeth virtual image contains an emphatically displayed mark, and the step of, according to the second relative position, synthesizing the personal virtual image and the intelligent-caring-device virtual image, to obtain the caring instructing video comprises:
according to the second relative position, determining a region of coincidence of a preset continuous duration between the teeth virtual image and the intelligent-caring-device virtual image as a target region;
deleting the emphatically displayed mark of the target region in the teeth virtual image, to obtain the teeth virtual image obtained after the deletion; and
according to the second relative position, synthesizing the personal virtual image containing the teeth virtual image obtained after the deletion and the intelligent-caring-device virtual image, to obtain the caring instructing video.

15. The method according to claim 12, wherein after the step of displaying the caring instructing video, the method further comprises:
collecting a caring image of the user;
acquiring a first position of the intelligent-caring-device image in the caring image of the user, and a second position of the intelligent-caring-device virtual image in the caring instructing video;
comparing the first position and the second position, to obtain a coinciding degree and a coinciding duration of the intelligent-caring-device image and the intelligent-caring-device virtual image; and
according to the coinciding degree and the coinciding duration, obtaining a caring score, and displaying the caring score.

16. An image displaying system, wherein the system comprises the intelligent caring mirror according to claim 1 and an intelligent caring device, and the intelligent caring device comprises:
a main toothbrush body; and
a second communication element configured for sending a caring record of a user to a first communication element of the intelligent caring mirror.

17. The image displaying system according to claim 16, wherein the main toothbrush body is provided with at least one identification mark at a surface.

18. A calculating and processing device, wherein the calculating and processing device comprises:
a memory storing a computer-readable code; and
one or more processors, wherein when the computer-readable code is executed by the one or more processors, the calculating and processing device implements the image displaying method according to claim 11.

19. A computer program product, wherein the computer program product comprises a computer-readable code, and when the computer-readable code is executed in a calculating and processing device, the computer-readable code causes the calculating and processing device to implement the image displaying method according to claim 11.

20. A non-transitory computer-readable storage medium, wherein the non-transitory computer-readable storage medium stores a computer instruction, and the computer instruction, when executed by a processor, implements the image displaying method according to claim 11.
